(19) United States
(12) Patent Application Publication
(10) Pub. No.: US 2012/0203119 A1
(43) Pub. Date: Aug. 9, 2012

(54) ELECTRONIC SPHYGMOMANOMETER

(75) Inventors: Shingo Yamashita, Kyoto-shi (JP); Yukiya Sawanoi, Nara-shi (JP)

(73) Assignee: OMRON HEALTHCARE CO., LTD., Kyoto (JP)

(21) Appl. No.: 13/368,628

(22) Filed: Feb. 8, 2012

Related U.S. Application Data
(63) Continuation of application No. PCT/JP2010/068263, filed on Oct. 18, 2010.

(30) Foreign Application Priority Data
Oct. 30, 2009 (JP) 2009-250484

(51) Int. Cl. A61B 5/022

(52) U.S. Cl. 600/490

(57) ABSTRACT

An electronic sphygmomanometer includes a cuff that can be worn at a measurement site, a pressure adjustment unit that adjusts the pressure applied inside the cuff; a pressure detection unit that includes multiple pressure sensors and that detects the cuff pressure inside the cuff based on pressure information output from the pressure sensors, a blood pressure calculation unit that calculates a blood pressure based on change in the cuff pressure detected by the pressure detection unit during blood pressure measurement, a keeping unit that keeps the cuff pressure at a predetermined pressure using the pressure adjustment unit during blood pressure measurement, and a sensor abnormality detection unit that, in a state in which the keeping unit keeps the cuff pressure at the predetermined pressure, detects whether an abnormality has occurred in the pressure sensors based on the pressure information output from the pressure sensors.

### TECHNICAL FIELD

The present invention relates to an electronic sphygmomanometer, and in particular to an electronic sphygmomanometer that improves the reliability of blood pressure measurement values.

### BACKGROUND ART

Blood pressure is one index for analyzing cardiovascular disease. Performing a cardiovascular disease risk analysis based on blood pressure is effective in preventing cardiovascular-related conditions such as stroke, heart failure, and myocardial infarction. In particular, early-morning hypertension; in which the blood pressure rises in the early morning, is related to heart disease, stroke, and the like. Among early-morning hypertension symptoms, the symptom called “morning surge”, in which the blood pressure rapidly rises within one hour to one and a half hours after waking up, has been found to have a causal relationship with stroke. In view of this, understanding the interrelationship between time (lifestyle) and changes in blood pressure is useful in risk analysis for cardiovascular-related conditions. It is therefore necessary to continuously measure blood pressure over a long period of time.

Also, it has been found in recent study results that home blood pressure measured at home is more effective in the prevention, diagnosis, treatment, etc. of cardiovascular-related conditions than blood pressure measured at a hospital or during a health examination (casual blood pressure). Accordingly, sphygmomanometers for home use have become widely prevalent, and home blood pressure values have started to be used in diagnosis.

In order to improve the measurement precision of sphygmomanometers,

In order to improve the measurement precision of sphygmomanometers, Patent Literature 1 (JP H7-51233A) discloses an invention in which processing for correcting error in a measurement value that is dependent on the characteristics of the pressure sensor for blood pressure measurement is performed in the electronic sphygmomanometer production stage.
Patent Literature 1: JP H7-51233A

### SUMMARY OF INVENTION

According to Patent Literature 1 (JP H7-51233β), the correction regarding the pressure sensor is performed based on differences in the characteristics of electronic sphygmomanometers in the electronic sphygmomanometer production stage. This kind of electronic sphygmomanometer is a sphygmomanometer for home use. Unlike a sphygmomanometer used in a medical facility such as a hospital, a sphygmomanometer for home use is generally not periodically corrected after purchase, except for certain situations such as a malfunction. For this reason, even if the pressure sensor output, which is of utmost importance in blood pressure measurement, deviates beyond the specified tolerance margin, there is no way to know that this has happened, and therefore it is not clear whether blood pressure measurement values are correct. Also, even if there is a large difference between a blood pressure measurement value and the normal blood pressure measurement value or the casual blood pressure measurement value, it is not clear whether the actual blood pressure values are different, or the blood pressure values are different due to error in the pressure sensor of the sphygmomanometer, thus causing the user to become concerned.

Also, some sphygmomanometers for medical facilities include two pressure sensors, and pressure is monitored based on the output of the two pressure sensors. However, the functions of these two pressure sensors are used for different purposes in such sphygmomanometers. Here, the blood pressure is calculated using cuff pressure information obtained by a first one of the pressure sensors, and abnormality detection is performed based on the output of the second pressure sensor. Specifically, an abnormality is detected if the pressure value detected by the second pressure sensor greatly exceeds 300 mmHg, for example. In this case, safety is ensured by stopping the pump and releasing the valve. Accordingly, the second pressure sensor is applied as a safety measure specified in the medical standard IEC 60601-2-30, and does not guarantee the precision of the first pressure sensor used for blood pressure measurement.

In view of this, one or more embodiments of the present invention provide an electronic sphygmomanometer that can improve the reliability of blood pressure measurement values in blood pressure measurement that employs multiple pressure sensors.

An electronic sphygmomanometer according to one or more embodiments of the present invention includes: a cuff that can be worn at a measurement site; a pressure adjustment unit that adjusts the pressure inside the cuff by pressurization or depressurization; a pressure detection unit that includes a plurality of pressure sensors and that detects the cuff pressure inside the cuff based on pressure information output from the plurality of pressure sensors; a blood pressure calculation unit that calculates a blood pressure based on change in the cuff pressure detected by the pressure detection unit at a time of blood pressure measurement; a keeping unit that keeps the cuff pressure at a predetermined pressure at the time of blood pressure measurement; and an abnormality detection unit that, in a state in which the keeping unit keeps the cuff pressure at the predetermined pressure, detects whether an abnormality has occurred in at least one of the plurality of pressure sensors based on the pressure information output from the plurality of pressure sensors.

According to one or more embodiments of the present invention, the blood pressure measurement includes a pressurization process in which the cuff is pressurized by the pressure adjustment unit after the blood pressure measurement has started, a depressurization process in which the cuff is depressurized, and a transition period from after the pressurization process is ended until when the depressurization process is started, and the keeping unit keeps the pressure applied in the cuff at the predetermined pressure in at least one of the pressurization process, the depressurization process, and the transition period.

According to one or more embodiments of the present invention, the predetermined pressure indicates the cuff pressure at the time when the pressurization process has ended.

According to one or more embodiments of the present invention, the abnormality detection unit includes a stabilization detection unit that detects whether the cuff pressure is being kept at the predetermined pressure based on the pressure information output from the plurality of pressure sensors, and in a case where the stabilization detection unit has detected that the cuff pressure is being kept at the predetermined pressure, the abnormality detection unit detects whether an abnormality has occurred in at least one of the plurality of pressure sensors based on the pressure information output from the plurality of pressure sensors.

According to one or more embodiments of the present invention, the stabilization detection unit detects, with respect to the pressure information output in time series from one of the plurality of pressure sensors, a difference in the pressure information at a plurality of time points, and detects whether the cuff pressure is being kept at the predetermined pressure based on the detected difference.

According to one or more embodiments of the present invention, the stabilization detection unit detects representative pressure information based on the pressure information output by the one of the pressure sensors at the plurality of time points, and, based on the representative pressure information, extracts, from among the pressure information at the plurality of time points output by at least one of the plurality of pressure sensors, the pressure information for detection of the difference.

According to one or more embodiments of the present invention, based on the pressure information at a plurality of time points that has been output by the plurality of pressure sensors in time series, the stabilization detection unit detects a difference in the pressure information at each of the time points, and detects whether the cuff pressure is being kept at the predetermined pressure based on a difference between the detected differences.

According to one or more embodiments of the present invention, the stabilization detection unit detects representative pressure information based on the pressure information output by the plurality of pressure sensors at the plurality of time points, and, based on the representative pressure information, extracts, from among the pressure information at the plurality of time points output by the plurality of pressure sensors, the pressure information for detection of the difference.

According to one or more embodiments of the present invention, the blood pressure measurement is stopped in a case where the abnormality detection unit has detected occurrence of an abnormality in at least one of the plurality of pressure sensors.

According to one or more embodiments of the present invention, the electronic sphygmomanometer further includes a storage unit, wherein each time the abnormality detection unit detects whether an abnormality has occurred in at least one of the plurality of pressure sensors, the storage unit stores a result of the detection, and when the blood pressure measurement is to be started, in a case where a determination has been made that the result of the detection that was read out from the storage unit indicates that an abnormality occurred, the blood pressure measurement is stopped, and the result of the detection that was read out is output.

According to one or more embodiments of the present invention, the electronic sphygmomanometer outputs a result of the detection performed by the abnormality detection unit.

According to one or more embodiments of the present invention, in a case where the abnormality detection unit has detected that an abnormality occurred in at least one of the plurality of pressure sensors, the blood pressure measurement is ended, and thereafter a result of the detection performed by the abnormality detection unit is output.

According to one or more embodiments of the present invention, the electronic sphygmomanometer further includes a data storage unit that stores blood pressure data indicating the blood pressure calculated by the blood pressure calculation unit and a result of the detection performed by the abnormality detection unit in association with the blood pressure data, wherein among the blood pressure data in the data storage unit, the blood pressure data that is associated with a detection result indicating the occurrence of an abnormality is excluded from the blood pressure data to be used for calculation of a statistic.

According to one or more embodiments of the present invention, in a case where the stabilization detection unit has not detected that the cuff pressure is being kept at the predetermined pressure, an alert of that fact is given.

According to one or more embodiments of the present invention, in a case where the stabilization detection unit has not detected that the cuff pressure is being kept at the predetermined pressure, the pressure adjustment unit pressurizes the cuff, and thereafter the stabilization detection unit again detects whether the cuff pressure is being kept at the predetermined pressure.

According to one or more embodiments of the present invention, in the process of blood pressure measurement performed based on cuff pressures detected using multiple pressure sensors, abnormality detection is performed with respect to at least one of the pressure sensors based on pressure information that was detected while the cuff pressures were held at a predetermined pressure. This enables performing accurate abnormality detection.

### BRIEF DESCRIPTION OF DRAWINGS

FIG. 1 is an external view of an electronic sphygmomanometer according to an embodiment.

FIG. 2 is a hardware configuration diagram of the electronic sphygmomanometer according to the embodiment.

FIG. 3 is a functional configuration diagram of the electronic sphygmomanometer according to the embodiment.

FIG. 4 is a diagram for illustrating blood pressure calculation according to the embodiment.

FIG. 5 is a graph showing the timing of pressure sensor abnormality detection during blood pressure measurement according to the embodiment.

FIG. 6 is a diagram for illustrating a stable period of a cuff pressure signal according to the embodiment.

FIGS. 7A to 7C are diagrams for illustrating a comparison of a reference value and an example of variation in differences between the output of the pressure sensors according to the embodiment.

FIGS. 8A and 8B are diagrams for illustrating a comparison of a reference value and another example of variation in differences between the output of the pressure sensors according to the embodiment.

FIG. 9 is a flowchart of processing for performing sensor abnormality detection at the end of a pressurization process in blood pressure measurement according to the embodiment.

FIG. 10 is a flowchart of processing for performing pressure sensor abnormality detection in the case of calculating a blood pressure in the pressurization process according to the embodiment.

FIG. 11 is a flowchart of other processing for performing pressure sensor abnormality detection in the case of calculating a blood pressure in the pressurization process according to the embodiment.

FIG. 12 is a flowchart of processing for performing sensor abnormality detection in the case of calculating a blood pressure in a depressurization process according to the embodiment.

FIG. 13 is a flowchart of other processing for performing sensor abnormality detection in the case of calculating a blood pressure in the depressurization process according to the embodiment.

FIG. 14 is a flowchart of processing for performing pressure sensor abnormality detection after the pressurization process or re-pressurization in the depressurization process according to the embodiment.

FIG. 15 is a flowchart of processing for retrying pressure sensor abnormality detection in the depressurization process according to the embodiment.

FIG. 16 is a diagram for illustrating an example of a display according to the embodiment.

FIG. 17 is an external view of a wrist-mounted electronic sphygmomanometer.

### DETAILED DESCRIPTION OF INVENTION

The following is a detailed description of an embodiment of the present invention with reference to the drawings. Note that like reference signs denote like or corresponding parts in the drawings, and redundant descriptions will not be given.

The present embodiment describes an electronic sphygmomanometer that includes multiple pressure sensors and performs oscillometric blood pressure calculation with respect to the upper arm as the measurement site. Note that the method applied for blood pressure calculation is not limited to the oscillometric method.

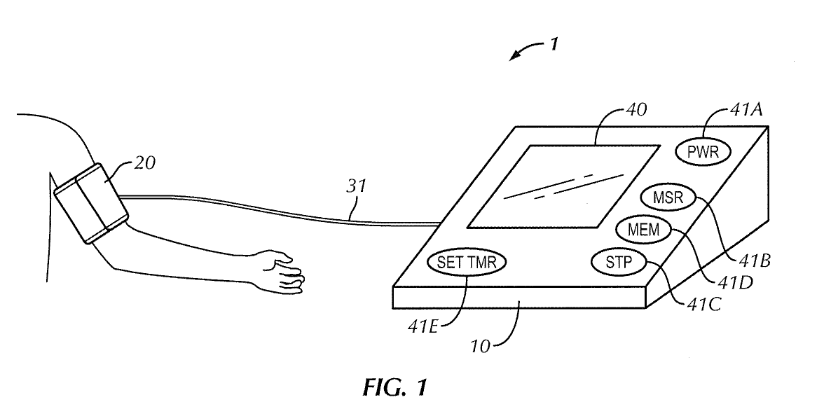

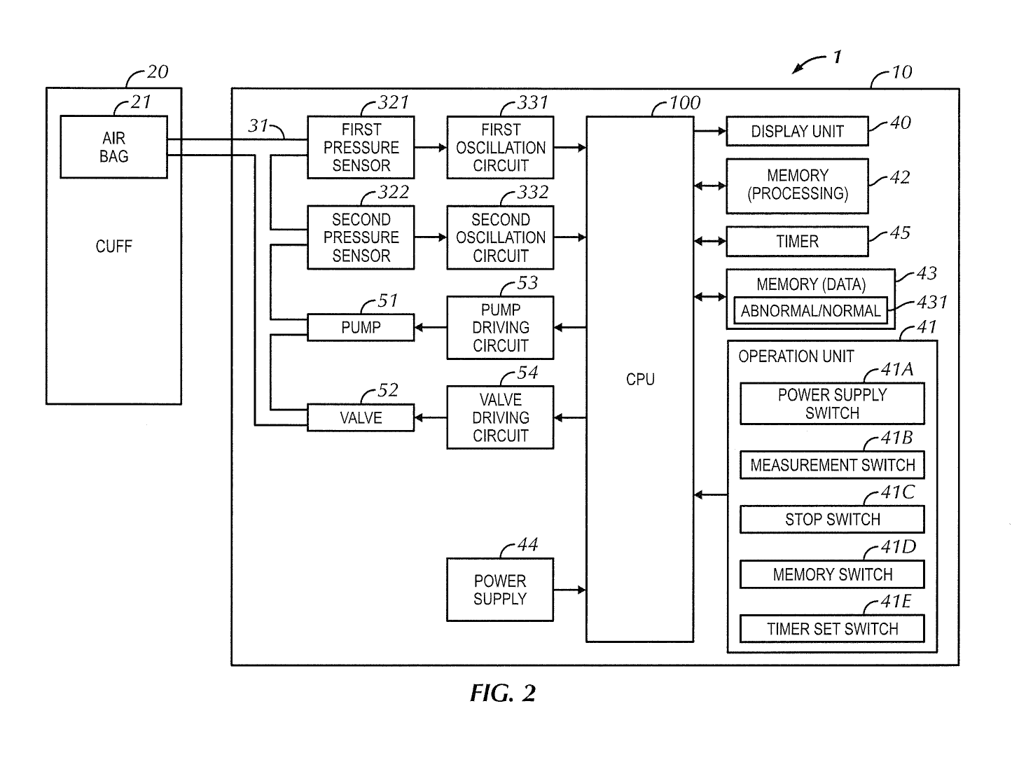

FIG. 1 is an external view of an electronic sphygmomanometer 1 according to this embodiment of the present invention, and FIG. 2 shows the hardware configuration of the electronic sphygmomanometer. As shown in FIGS. 1 and 2, the electronic sphygmomanometer 1 includes a main body unit 10 and a cuff 20 that can be wrapped around the upper arm of a measurement subject. The cuff 20 includes an air bag 21. Disposed on the surface of the main body unit 10 are a display unit 40 that is configured by a liquid crystal display or the like, and an operation unit 41 that is made up of multiple switches for receiving instructions from a user (measurement subject).

In addition to the display unit 40 and the operation unit 41 described above, the main body unit 10 includes a CPU (Central Processing Unit) 100 for performing central control of various units and performing various types of arithmetic processing, a processing memory 42 for storing data and programs for causing the CPU 100 to perform predetermined operations, a data storage memory 43 for storing measured blood pressure data and the like, a power supply 44 for supplying power to various units in the main body unit 10, and a timer 45 for measuring the current time and outputting time data to the CPU 100.

The operation unit 41 has a power supply switch (“PWR”) 41A for receiving the input of an instruction for switching the power supply on or off, a measurement switch (“MSR”) 41B for receiving the input of a measurement start instruction, a stop switch (“STP”) 41C for receiving the input of a measurement stop instruction, a memory switch (“MEM”) 41D for receiving the input of an instruction for causing information such as blood pressure data stored in the memory 43 to be read out from the memory 43 and displayed by the display unit 40, and a timer set switch (“SET TMR”) 41E that is operated in order to set the timer 45.

The main body unit 10 furthermore has a cuff pressure adjustment mechanism that includes a pump 51 and a discharge valve (hereinafter, simply referred to as the “valve”) 52.

An air system is made up of the pump 51, the valve 52, and first and second pressure sensors 321 and 322 for detecting the pressure (cuff pressure) in the air bag 21, and the air system is connected to the air bag 21, which is enclosed in the cuff 20, via an air tube 31.

In addition to the air system and cuff pressure adjustment mechanism that are described above, the main body unit 10 furthermore includes first and second oscillation circuits 331 and 332. The cuff pressure adjustment mechanism includes a pump driving circuit 53 and a valve driving circuit 54 in addition to the pump 51 and the valve 52.

The pump 51 is driven in order to increase the cuff pressure. When the pump 51 is driven, air is supplied to the air bag 21. By opening or closing the valve 52, air is discharged from the air bag 21, or air is enclosed inside the air bag 21. The pump driving circuit 53 controls the pump 51 based on a control signal transmitted from the CPU 100. The valve driving circuit 54 controls the valve 52 based on a control signal transmitted from the CPU 100. Accordingly, the pump 51 is controlled based on a control signal so as to be driven or stopped by the pump driving circuit 53, and the valve 52 is controlled based on a control signal so as to be opened or closed by the valve driving circuit 54.

The first and second pressure sensors 321 and 322 are capacitive pressure sensors in which the capacitance value changes according to the cuff pressure that is detected. The first and second oscillation circuits 331 and 332 are, respectively, connected to corresponding pressure sensors and oscillate based on the capacitance values of the corresponding pressure sensors. Accordingly, the first and second oscillation circuits 331 and 332 each output, to the CPU 100, a signal having a frequency that corresponds to the capacitance value of the corresponding pressure sensor (hereinafter, referred to as a “frequency signal”). The CPU 100 performs pressure detection by converting the frequency signals input from the first oscillation circuit 331 and the second oscillation circuit 332 into a pressure. Here, the CPU 100 is assumed to alternately input the frequency signals from the first oscillation circuit 331 and the second oscillation circuit 332 at staggered times.

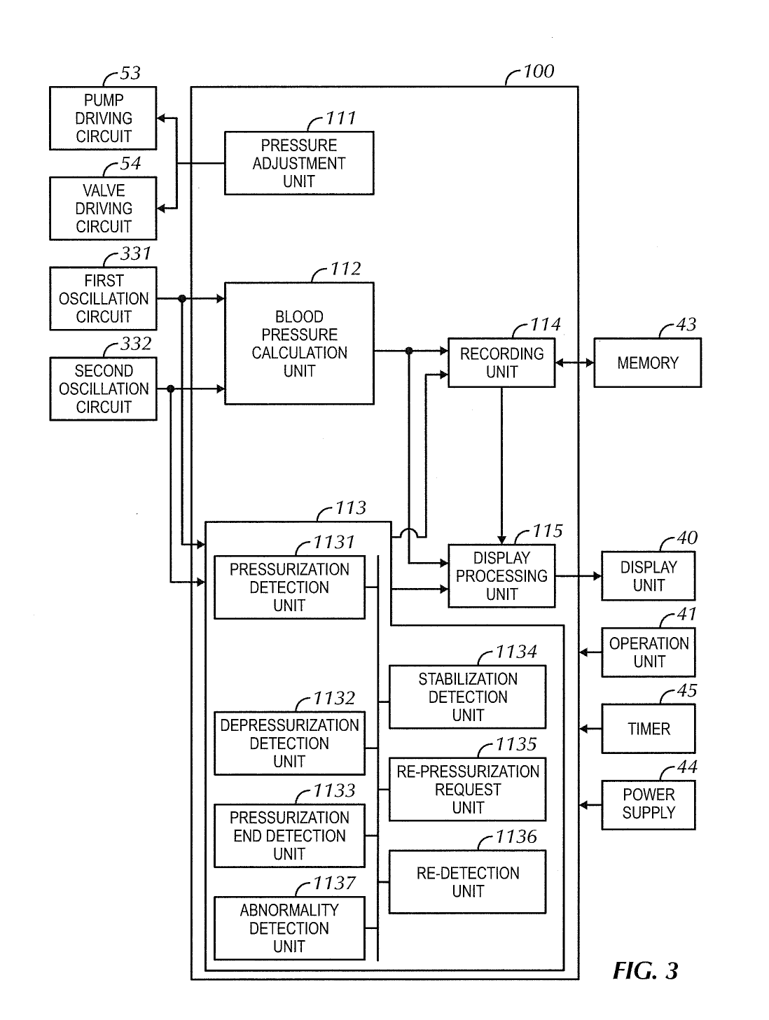

FIG. 3 shows the functional configuration of the electronic sphygmomanometer 1. As shown in FIG. 3, the CPU 100 includes a pressure adjustment unit 111, a blood pressure calculation unit 112, a sensor abnormality detection unit 113, a recording unit 114, and a display processing unit 115.

The pressure adjustment unit 111 controls the pump 51 and the valve 52 via the pump driving circuit 53 and the valve driving circuit 54 so as to cause air to flow into the air bag 21 or be discharged from the air bag 21 via the air tube 31. In this way, the pressure adjustment unit 111 adjusts the cuff pressure. It is assumed that some or all of the functions of these units are realized by the CPU 100 reading out corresponding programs and data from the memory 42 and executing commands described therein.

The blood pressure calculation unit 112 detects pulse wave amplitude information based on a frequency signal input from the first oscillation circuit 331 or the second oscillation circuit 332 (the frequency signal indicating a pressure information signal), calculates a systolic blood pressure SYS corresponding to the maximum blood pressure and a diastolic blood pressure DIA corresponding to the minimum blood pressure based on the detected pulse wave amplitude information in accordance with the oscillometric method, as well as calculates a number of beats per predetermined time based on the detected pulse wave amplitude information. Specifically, in the process in which the pressure adjustment unit 111 gradually pressurizes (or depressurizes) the cuff pressure to a predetermined value, the blood pressure calculation unit 112 detects pulse wave amplitude information based on the cuff pressure input from first oscillation circuit 331 or the second oscillation circuit 332, and calculates the systolic blood pressure and the diastolic blood pressure of the measurement subject based on the detected pulse wave amplitude information. A conventionally known method can be applied in the blood pressure calculation and the pulse calculation performed by the blood pressure calculation unit 112 in accordance with the oscillometric method.

The sensor abnormality detection unit 113 receives an input of frequency signals output from the first oscillation circuit 331 and the second oscillation circuit 332, and performs abnormality detection with respect to the first pressure sensor 321 and the second pressure sensor 322 by analyzing the input signals.

The sensor abnormality detection unit 113 has a pressurization detection unit 1131 for performing abnormality detection in the cuff pressure pressurization process, a depressurization detection unit 1132 for performing abnormality detection in the cuff pressure depressurization process, a pressurization end detection unit 1133 for performing abnormality detection when the pressurization process ends, a stabilization detection unit 1134 for detecting the fact that the cuff pressure detected in abnormality detection has stabilized, a re-pressurization request unit 1135 for requesting re-pressurization in the case where the cuff pressure has not stabilized, a re-detection unit 1136 for re-performing abnormality detection in the case where the cuff pressure has not stabilized, and an abnormality detection unit 1137 for performing pressure sensor abnormality detection based on a result of comparing cuff pressures and a reference value.

The recording unit 114 has the functions of reading out data from the memory 43 and writing data to the memory 43. Specifically, the recording unit 114 receives an input of output data from the blood pressure calculation unit 112 and stores the input data (blood pressure measurement data) in a predetermined storage area of the memory 43. The recording unit 114 furthermore receives an input of output data from the sensor abnormality detection unit 113 and stores the input data (pressure sensor abnormality detection result) in a predetermined storage area of the memory 43. Also, based on an operation performed on the memory switch 41D of the operation unit 41, the recording unit 114 reads out measurement data from a predetermined storage area of the memory 43 and outputs the read-out data to the display processing unit 115.

The display processing unit 115 receives an input of data, converts the input data into a displayable format, and displays the data on the display unit 40.

Note that with regard to peripheral circuits of the CPU 100, FIG. 3 only shows parts that make direct exchanges with the CPU 100.

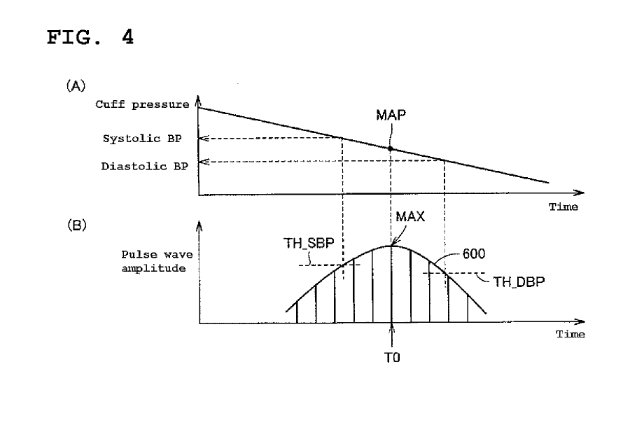

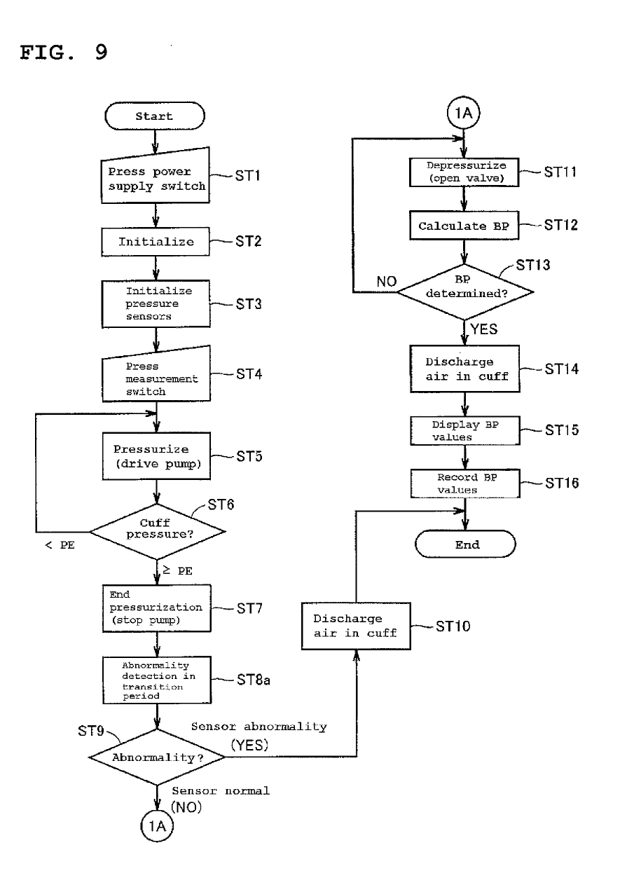

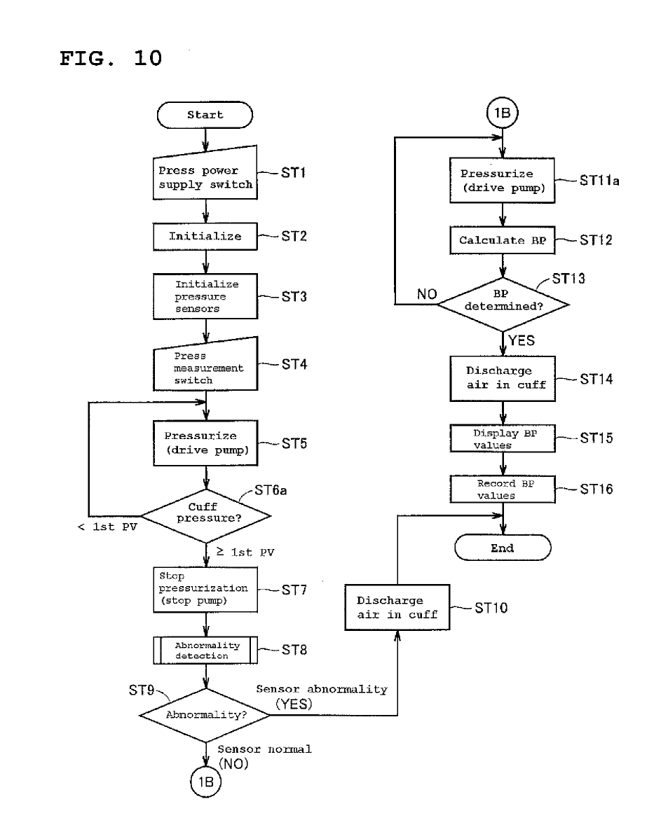

Next is a description of operations of various units with reference to FIGS. 4 to 18. The flowcharts of FIGS. 9 to 15 are stored in advance as programs in the memory 42, and the processing of various units is realized by the CPU 100 reading out the programs from the memory 42 and executing the read-out programs.

(Blood Pressure Calculation Procedure)

The following describes the concept of an oscillometric blood pressure calculation method according to the present embodiment. In (A) of FIG. 4, gradually decreasing cuff pressures are shown along an axis of time measured by the timer 45. In (B) of FIG. 4, a pulse wave amplitude envelope 600 corresponding to the aforementioned pulse wave amplitude information is shown along the same time axis. The pulse wave amplitude envelope 600 is detected by a pulse wave amplitude signal superimposed on a signal (cuff pressure) from a pressure sensor being extracted in time series.

As shown in (A) and (B) of FIG. 4, upon detecting a maximum amplitude value MAX in the pulse wave amplitude envelope 600, the blood pressure calculation unit 112 calculates two threshold values TH_DBP and TH_SBP by multiplying that maximum value by predetermined constants (e.g., 0.7 and 0.5). The cuff pressure at the intersection between the threshold value TH_DBP and the envelope 600 on the low cuff pressure side of a cuff pressure MAP (average blood pressure) at time T0 at which the maximum value MAX was detected, is then calculated as the diastolic blood pressure DIA. Also, the cuff pressure at the intersection between the threshold value TH_SBP and the envelope 600 on the high cuff pressure side of the cuff pressure MAP is then calculated as the systolic blood pressure SYS.

Although blood pressure calculation in the depressurization process has been described above, it is possible to detect the pulse wave amplitude envelope 600 and calculate the systolic blood pressure SYS and the diastolic blood pressure DIA using a similar procedure in the pressurization process as well.

(Sensor Abnormality Determination Method)

In order to improve the reliability of blood pressure measurement values, the sensor abnormality detection unit 113 performs abnormality detection in the following way in the blood pressure measurement process. Specifically, the frequency signals input from the first and second oscillation circuits 331 and 332 are converted into cuff pressures a and b, respectively, and the cuff pressure a and the cuff pressure b that were obtained by conversion are compared with a later-described reference value β (e.g., 5 mmHg). Based on the comparison result, a determination that an abnormality has occurred in one of the pressure sensors is made in the case where the difference between the cuff pressure a and the cuff pressure b exceeds the reference value β.

Also, in the case where three or more pressure sensors are used, the difference is calculated between the maximum value and the minimum value among the three or more cuff pressures similarly obtained by conversion, and a determination that an abnormality has occurred in any one of the pressure sensors is made in the case where the calculated difference exceeds the reference value β.

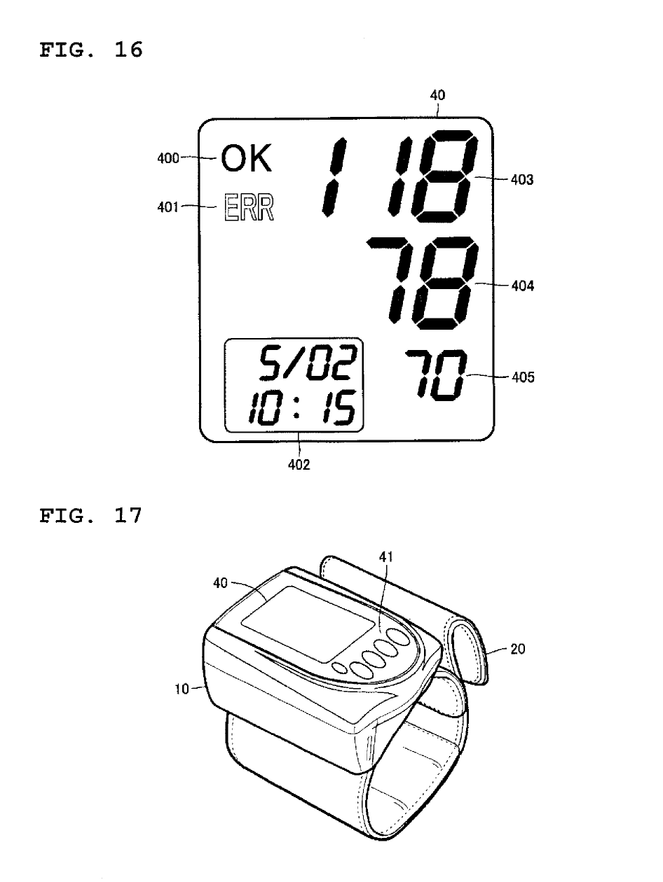

In the case where the sensor abnormality detection unit 113 has determined that an abnormality has occurred in any one of the pressure sensors, the blood pressure calculation unit 112 does not use the calculated blood pressure measurement data in display or recording (i.e., discards the calculated blood pressure measurement data) based on the determination result, thus enabling improving the reliability of blood pressure measurement values. Also, instead of discarding the blood pressure measurement data, a configuration is possible in which the display unit 40 displays the blood pressure measurement data along with information (a message) indicating that an abnormality has occurred in a pressure sensor (see FIG. 16, which is described later). Also, a configuration is possible in which such blood pressure measurement data is stored in the memory 43 in association with a flag indicating that an abnormality has occurred in a pressure sensor.

In the case where blood pressure measurement data in the memory 43 is used in order to calculate a statistic for determining whether the blood pressure of the measurement subject belongs to the high blood pressure category, for example, a configuration is possible in which blood pressure measurement data that has been associated with the aforementioned flag among the blood pressure measurement data stored in the memory 43 is excluded from data targeted for use in the calculation of the statistic.

Also, each time the sensor abnormality detection unit 113 performs the detection operation, data 431 indicating the detection result (abnormality/normal) is stored in a predetermined area of the memory 43 by overwriting. Then, a configuration is possible in which the CPU 100 reads out the data 431 from the memory 43 when the start of blood pressure measurement is instructed by the switch 41B being operated, blood pressure measurement is stopped and the read-out data 431 is displayed on the display unit 40 if it has been determined that the read-out data 431 indicates an abnormality, and pressurization for blood pressure measurement is started if it has been determined that the read-out data 431 does not indicate an abnormality (i.e., indicates normal operation).

(Timing of Pressure Sensor Abnormality Detection)

An advantage of the present embodiment is that because the step in which the sensor abnormality detection unit 113 performs pressure sensor abnormality detection is carried out in the blood pressure measurement process, there is no need to provide a separate abnormality detection step.

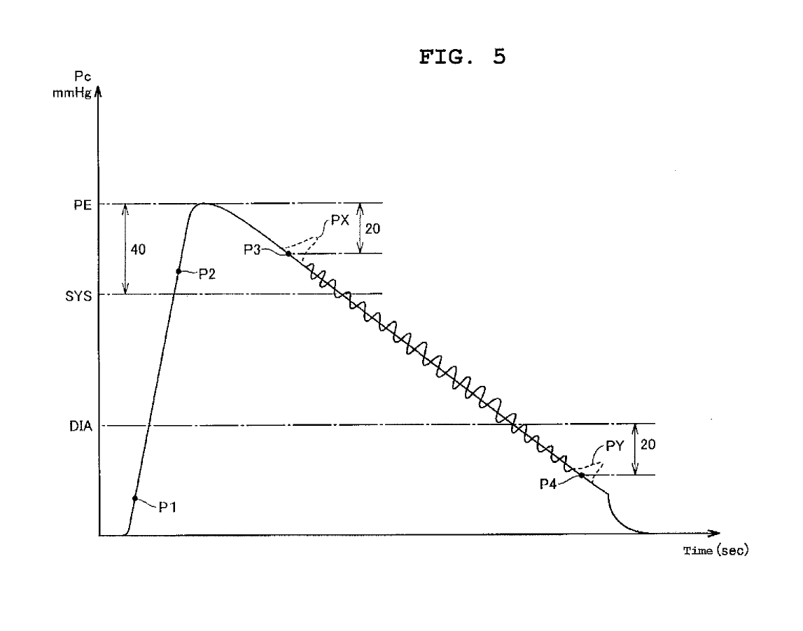

FIG. 5 schematically shows change in a cuff pressure Pc over time during blood pressure measurement. In blood pressure measurement, after the cuff 20 has been wrapped around the measurement site, pressurization is started in response to an operation being performed on the measurement switch 41B. When pressurized is started, the cuff pressure Pc gradually rises, and pressurization is carried out until the cuff pressure Pc reaches a pressurization end pressure PE. This is referred to as the pressurization process.

After the pressurization end pressure PE has been reached, discharge is started. Specifically, a transition to the depressurization process occurs due to the valve 52 being opened so that the air inside the cuff 20 is gradually discharged. The diastolic blood pressure DIA and the systolic blood pressure SYS are detected (calculated) in the depressurization process as well.

In the present embodiment, pressure sensor abnormality detection is executed in both the pressurization process and the depressurization process. Specifically, pressure sensor abnormality detection is carried out when a cuff pressure Pc lower than the diastolic blood pressure DIA has been detected (see pressures P1 and P4 in FIG. 5), and when a cuff pressure Pc that is higher than the systolic blood pressure SYS and lower than the pressurization end pressure PE has been detected (see pressures P2 and P3 in FIG. 5). Furthermore, pressure sensor abnormality detection is carried out in the period from the end of the pressurization process to the start of the depressurization process. In the present embodiment, it is assumed that pressure sensor abnormality detection is carried out at least any one or more of these times and this period.

Here, it is assumed that the pressurization end pressure PE is a value that is 40 mmHg greater than the systolic blood pressure SYS, the pressure P3 is a value that is 20 mmHg less than the pressurization end pressure PE, and the pressure P4 is a value that is 20 mmHg less than the diastolic blood pressure DIA.

(Detection of Stabilization of Cuff Pressure Pc)

In the present embodiment, when pressure sensor abnormality detection is to be performed, the cuff pressure is controlled so as to be constant, and abnormality detection is carried out if the stabilization detection unit 1134 has detected that the cuff pressure is constant. This enables maintaining precision in abnormality detection.

The following describes processing in which the stabilization detection unit 1134 detects that the cuff pressure is constant, taking the example of the period from the end of the pressurization process until the start of transition to the depressurization process (hereinafter, this period is referred to as the “transition period”), with reference to FIGS. 5 to 7C.

The stabilization detection unit 1134 detects the transition period based on an output signal from the pressure adjustment unit 111. Specifically, the stabilization detection unit 1134 detects the period from when the pressure adjustment unit 111 stops the pump 51 (i.e., ends the pressurization process) by outputting a stop signal to the pump driving circuit 53 until when the pressure adjustment unit 111 thereafter opens the closed valve 52 (i.e., starts the transition to the depressurization process) by outputting a signal to the valve driving circuit 54. In the transition period, the pump 51 is stopped, and the valve 52 is fully closed, and therefore the cuff pressure is constant.

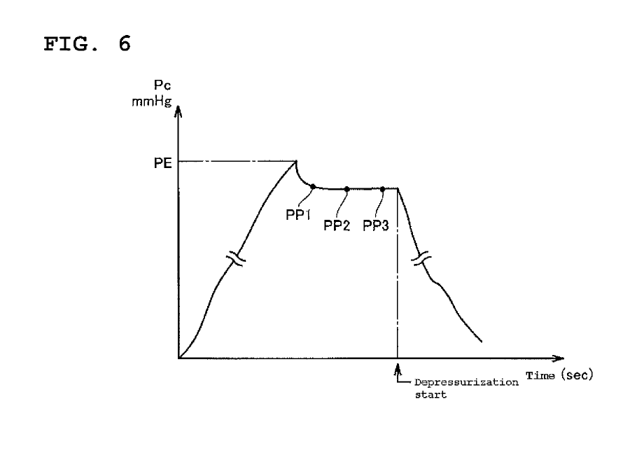

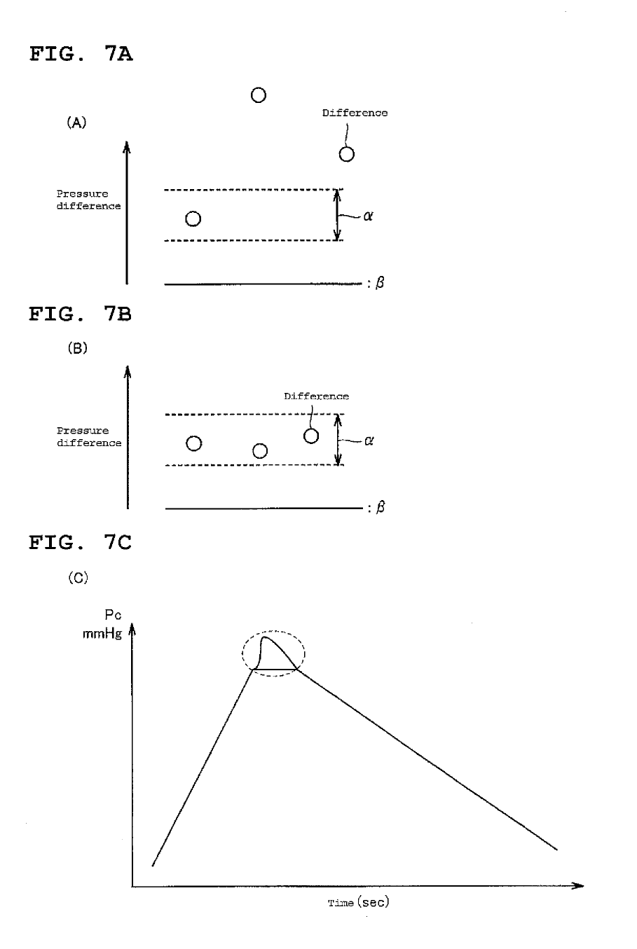

In the transition period, at a predetermined interval based on time data from the timer 45 (at the mutually different times PP1, PP2, and PP3 in FIG. 6), the stabilization detection unit 1134 calculates the cuff pressure detected by the first pressure sensor 321 based on an input signal from the first oscillation circuit 331, and subsequently calculates the cuff pressure detected by the second pressure sensor 322 based on an input signal from the second oscillation circuit 332. The difference between the detected cuff pressures of the first and second pressure sensors is then detected for each timing. The difference between the differences detected at the respective timings is then compared with a threshold value a (see FIGS. 7A and 7B) that has been read out from the memory 43. The threshold value a is an allowable range value for allowing or prohibiting the pressure sensor abnormality detection operation, that is to say, the threshold value a indicates whether the cuff pressure is stable. Accordingly, as a result of the comparison, in the case where it has been determined that the difference between the differences detected at the respective timings does not exceed the allowable range indicated by the threshold value α (see FIG. 7B), it is detected that the cuff pressure is constant in the transition period. In the case where it has been detected that the cuff pressure is constant, pressure sensor abnormality detection processing is started.

On the other hand, in the case where the cuff pressure fluctuates due to, for example, body movement of the measurement subject in the transition period (portion enclosed in a broken line in FIG. 7C), the difference between the differences exceeds the allowable range indicated by the threshold value α (see FIG. 7A), it is detected that the cuff pressure is not stable, and pressure sensor abnormality detection processing is not started.

Note that even in the case where it has been detected that the cuff pressure is not stable, it is possible for the stabilization detection unit 1134 to again perform cuff pressure stabilization detection.

The above-described detection of cuff pressure stabilization by the stabilization detection unit 1134 is carried out using the same procedure at each of the pressures P1, P2, P3, and P4 in FIG. 5.

Although cuff pressure stabilization detection is performed using the cuff pressures of both the first pressure sensor 321 and the second pressure sensor 322 in the above description, a configuration is possible in which stabilization detection is performed using cuff pressures detected by either one of the pressure sensors. Specifically, a configuration is possible in which the cuff pressure of one of the pressure sensors is detected at each of the times PP1, PP2, and PP3, and it is detected that the cuff pressure is stable in the case where the difference between the cuff pressures detected at the respective times does not exceed a predetermined value.

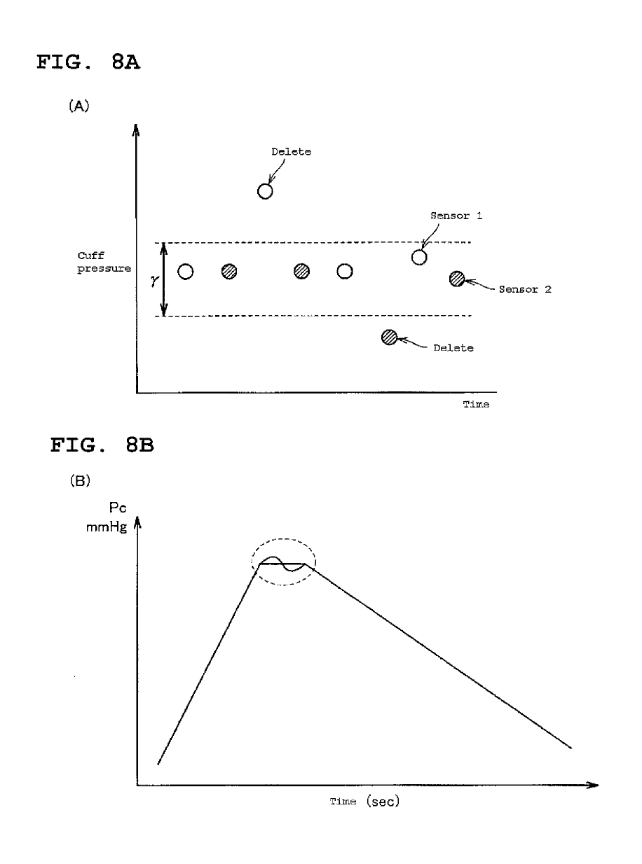

The following describes processing that may be performed in order to improve precision in the above-described detection of stabilization, with reference to FIGS. 8A and 8B.

As shown in FIG. 8B, even in the transition period, in the case where amplitude fluctuation (see the portion enclosed in a broken line in FIG. 8B) resulting from a large disturbance (body movement or pulse wave) is superimposed on the cuff pressure signal, there is a large amount of fluctuation in the values of the cuff pressures detected by the first pressure sensor 321 and the second pressure sensor 322. In view of this, the stabilization detection unit 1134 calculates an average value as a representative value for all of the cuff pressures that are input from the first and second pressure sensors 321 and 322 in time series. The values of each of the cuff pressures detected by the first pressure sensor 321 and the second pressure sensor 322 are then compared with a threshold value γ indicating a predetermined range including the average value. Whether the cuff pressure values fall within the threshold value γ is detected based on the comparison results. As a result of the detection, any cuff pressure values that have been determined to fall outside the threshold value γ are excluded from the cuff pressure values shown in FIG. 8A that are to be referenced for stabilization detection. Accordingly, extremely high and low cuff pressures are excluded from the reference values to be used in stabilization detection. Accordingly, cuff pressure values that are to be referenced for stabilization detection, that is to say, cuff pressure values that are to be used in order to detect differences, can be selectively extracted, based on the representative value, from among the cuff pressure values output by the first pressure sensor 321 and the second pressure sensor 322 at multiple points in time. This consequently enables improving precision in stabilization detection.

Although an average value is detected as the representative value in the above description, a median value may be used.

Although the representative value is detected using the cuff pressures of both the first pressure sensor 321 and the second pressure sensor 322 in the above description, a configuration is possible in which the representative value is detected using cuff pressures detected by either one of the pressure sensors. In this case, the representative value is used to exclude extremely high and low cuff pressure values from among the cuff pressure values detected by the one pressure sensor in time series. Accordingly, cuff pressure values that are to be referenced for stabilization detection, that is to say, cuff pressure values that are to be used in order to detect differences, can be selectively extracted, based on the representative value, from among the cuff pressure values output by the one pressure sensor at multiple points in time. This consequently enables improving precision in stabilization detection.

(Pressure Sensor Abnormality Detection)

In the above-described transition period, in the case where stabilization of the cuff pressure is detected, the pressurization end detection unit 1133 compares each of the difference values within the range of the threshold value α with the reference value β (see FIGS. 7A and 7B) read out from the memory 43. Note that in the case where stabilization has been detected using only one pressure sensor, when abnormality detection is performed, values from both of the pressure sensors are detected and used in the comparison. Based on the result of the comparison, in the case where it has been detected that all of the difference values exceed the reference value β, it is detected that an abnormality has occurred in at least either the first pressure sensor 321 or the second pressure sensor 322. Here, the reference value β indicates a difference threshold value for detecting an abnormality such as malfunctioning of the first and second pressure sensors 321 and 322.

Here, it is assumed that the threshold values a and 13 have been detected in advance through experimentation or the like.

Re-Pressurization and Re-Detection

In the depressurization process (before blood pressure calculation) and the pressurization process, which are not in the transition period, in the case where the stabilization detection unit 1134 detects that the cuff pressure has not stabilized due to, for example, body movement of the measurement subject when pressure sensor abnormality detection is to be performed, the pressure adjustment unit 111 starts rotation (driving) of the pump 51 via the pump driving circuit 53 in response to the detection signal. Accordingly, the cuff pressure is increased again. Thereafter, the stabilization detection unit 1134 performs cuff pressure stabilization detection again. Specifically, in the case where amplitude fluctuation (see the broken line PX in FIG. 5) resulting from body movement is detected at the cuff pressure P3 in the depressurization process (before blood pressure calculation), re-pressurization is performed, and thereafter cuff pressure stabilization detection is performed again.

Also, when pressure sensor abnormality detection is to be performed after blood pressure calculation has ended in the depressurization process, in the case where the stabilization detection unit 1134 has detected that the cuff pressure has not stabilized due to, for example, body movement of the measurement subject, the pressure adjustment unit 111 reduces the cuff pressure to a predetermined pressure in response to the detection signal by opening the closed valve 52 via the valve driving circuit 54. Thereafter, the stabilization detection unit 1134 performs cuff pressure stabilization detection again. Specifically, in the case where amplitude fluctuation (see the broken line PY in FIG. 5) resulting from body movement is detected at the time P4 in the depressurization process (after blood pressure calculation), depressurization is performed, and thereafter cuff pressure stabilization detection is performed again.

Note that although re-pressurization and re-detection are executed in order to perform stabilization detection again in the above description, in the case where stabilization is not detected, this fact may be displayed by the display unit 40 or the like in order to prevent body movement. This enables presenting a message for prompting the measurement subject to be still.

(Blood Pressure Measurement Processing)

The following describes blood pressure measurement processing in different cases of timings according to which pressure sensor abnormality detection is performed.

Pressure Sensor Abnormality Detection at End of Pressurization Process

Below is a description of a procedure for carrying out pressure sensor abnormality detection in the above-described transition period with reference to FIG. 9.

First, if the measurement subject operates (presses) the power supply switch 41A (step ST1), the CPU 100 initializes a work memory that is not shown (step ST2).

Next, the first and second pressure sensors 321 and 322 are adjusted to 0 mmHg (step ST3).

Here, the measurement subject wraps the cuff 20 around the measurement site as shown in FIG. 1. After the cuff 20 has been wrapped around the measurement site, if the measurement subject operates (presses) the measurement switch 41B (step ST4), the pressure adjustment unit 111 outputs control signals to the pump driving circuit 53 and the valve driving circuit 54. Based on the control signals, the pump driving circuit 53 and the valve driving circuit 54 close the valve 52 and thereafter drive the pump 51. Accordingly, the pressure adjustment unit 111 compares the cuff pressure detected by the first pressure sensor 321 with the pressurization end pressure PE read out from the memory 42, and gradually increases the cuff pressure to the pressurization end pressure PE based on the comparison results (steps ST5 and ST6).

After the cuff pressure has been increased to the pressurization end pressure PE (“≧PE” in step ST6), the pressure adjustment unit 111 outputs control signals to the pump driving circuit 53 and the valve driving circuit 54. Based on the control signals, the pump driving circuit 53 and the valve driving circuit 54 stop the pump 51 and close the valve 52 (step ST7). Accordingly, the cuff pressure is kept constant in the transition period.

Next, the stabilization detection unit 1134 detects whether the cuff pressure has stabilized as described above in the transition period. If it has been detected that the cuff pressure has not stabilized, and the pressurization end detection unit 1133 has detected that a pressure sensor abnormality has occurred (step ST8a and YES in step ST9), the pressure adjustment unit 111 fully opens the valve 52 via the valve driving circuit 54 (step ST10). Accordingly, air is rapidly discharged from the cuff 20, and this series of processing ends.

While the cuff pressure is stable, if the pressurization end detection unit 1133 determines that a pressure sensor abnormality has not occurred (NO in step ST9), the blood pressure is calculated in the depressurization process.

Specifically, the pressure adjustment unit 111 gradually opens the valve 52 via the valve driving circuit 54. The cuff pressure therefore gradually decreases (step ST11).

In this depressurization process, the blood pressure calculation unit 112 detects pulse wave amplitude information based on the frequency signals output by the first oscillation circuit 331 and the second oscillation circuit 332, that is to say, based on the cuff pressure signals detected by the first pressure sensor 321 and the second pressure sensor 322, and performs a predetermined arithmetic operation on the detected pulse wave amplitude information. The systolic blood pressure SYS and the diastolic blood pressure DIA are calculated by this arithmetic operation (steps ST12 and ST13). The pulse wave amplitude information represents a volume change distribution with respect to the artery at the measurement site and is included in the detected cuff pressure signals.

If the systolic blood pressure SYS and the diastolic blood pressure DIA are calculated, and blood pressure values are determined (YES in step ST13), the pressure adjustment unit 111 fully opens the valve 52 via the valve driving circuit 54. Accordingly, the air in the cuff 20 is rapidly discharged (step ST14).

The blood pressure data calculated by the blood pressure calculation unit 112 is output to the display processing unit 115 and the recording unit 114. The display processing unit 115 receives an input of the blood pressure data, and displays the input blood pressure data on the display unit 40 (step ST15). Also, the recording unit 114 receives an input of the blood pressure data, and stores the input blood pressure data in a predetermined storage area of the memory 43 in association with time data that has been input from the timer 45 (step ST16).

Note that the blood pressure calculation unit 112 can also calculate a pulse rate based on the detected pulse wave amplitude information. The calculated pulse rate is displayed on the display unit 40 by the display processing unit 115, and stored in the memory 43 by the recording unit 114 in association with the blood pressure data.

Also, in the case where it has been determined that a sensor abnormality has occurred (YES in step ST9), the blood pressure is not calculated, and therefore the message “Sensor trouble” may be displayed on the display unit 40. Based on the displayed message, the measurement subject can confirm that the blood pressure was not calculated due to a sensor abnormality.

In the flowchart of FIG. 9, in the case where a pressure sensor abnormality has been detected, blood pressure measurement processing is stopped (blood pressure calculation is not performed), but a configuration is possible in which blood pressure measurement processing is continued instead of being stopped, and the message “Sensor trouble” is displayed on the display unit 40 after the blood pressure measurement.

Pressure Sensor Abnormality Detection at Cuff Pressure P1 in FIG. 5

Below is a description of a procedure for carrying out pressure sensor abnormality detection at a cuff pressure (cuff pressure P1 in FIG. 5) that is lower than the diastolic blood pressure DIA in the pressurization process, with reference to FIG. 10. It is assumed that the value of the cuff pressure P1 (first pressure value) is stored in the memory 42 in advance.

In FIG. 10, the processing of steps ST1 to ST4 is performed in the same manner as the corresponding steps in FIG. 9. Next, the pressure adjustment unit 111 compares the cuff pressure detected by the first pressure sensor 321 with the cuff pressure P1 read out from the memory 42, and continues performing pressurization by rotating the pump 51 until, based on the comparison results, the cuff pressure is greater than or equal to the first pressure value (i.e., the value of the cuff pressure P1) (“≧1st PV” in step ST6a). Thereafter, the pump 51 is stopped. The cuff pressure is therefore kept constant (step ST7).

Next, the stabilization detection unit 1134 detects whether the cuff pressure has stabilized as described above. If the cuff pressure has not stabilized, and the pressurization detection unit 1131 has detected that a pressure sensor abnormality has occurred (step ST8 and YES in step ST9), the pressure adjustment unit 111 fully opens the valve 52 via the valve driving circuit 54 (step ST10). Accordingly, air is rapidly discharged from the cuff 20, and this series of processing ends. In this way, blood pressure measurement processing is stopped in the case where a pressure sensor abnormality has been detected. Note that details of abnormality detection processing (step ST8) will be described later.

While the cuff pressure is stable, if the pressurization detection unit 1131 determines that a pressure sensor abnormality has not occurred (NO in step ST9), the pressurization process continues until the cuff pressure reaches the pressurization end pressure PE, and the blood pressure is calculated during that time (steps ST11a and ST12).

If the systolic blood pressure SYS and the diastolic blood pressure DIA have been determined (YES in step ST13), processing is performed in the same manner as the processing of steps ST14 to ST16 in FIG. 9.

Pressure Sensor Abnormality Detection at Cuff Pressure P2 in FIG. 5

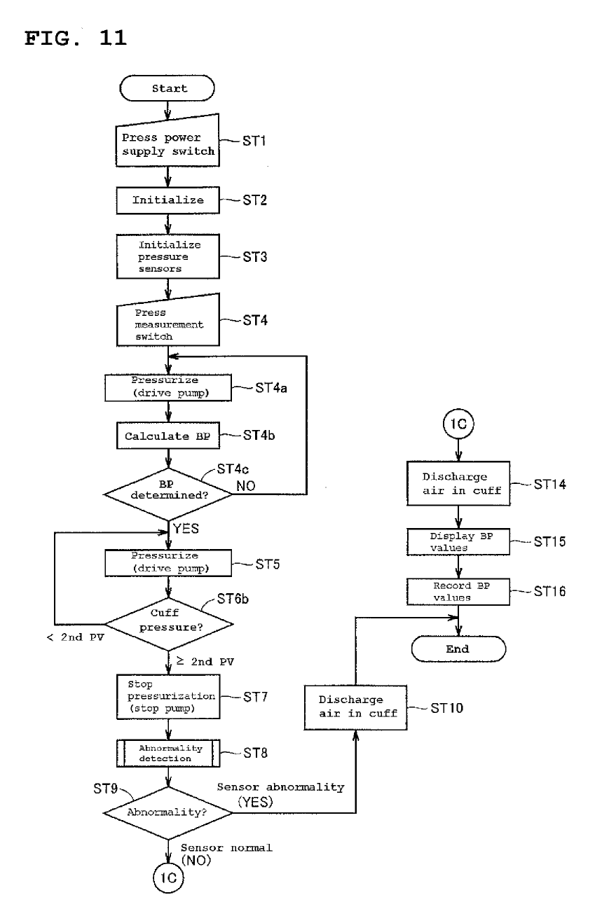

Below is a description of a procedure for carrying out pressure sensor abnormality detection at a cuff pressure (cuff pressure P2 in FIG. 5) that is higher than the systolic blood pressure SYS in the pressurization process, with reference to FIG. 11. It is assumed that the value of the cuff pressure P2 (second pressure value) is stored in the memory 42 in advance.

In FIG. 11, the processing of steps ST1 to ST4 is performed in the same manner as the corresponding steps in FIG. 9. Next, the pump 51 rotates so as to raise the cuff pressure, and blood pressure calculation is performed in the pressurization process (steps ST4a to ST4c). If the blood pressure values (diastolic blood pressure DIA and systolic blood pressure SYS) have been determined, the pressure adjustment unit 111 compares the cuff pressure detected by the first pressure sensor 321 with the cuff pressure P2 read out from the memory 42, and continues performing pressurization by rotating the pump 51 until, based on the comparison results, it has been determined that the cuff pressure is greater than or equal to the second pressure value (i.e., the value of the cuff pressure P2) (“≧2nd PV” in step ST6b). Thereafter, the pump 51 is stopped, and the cuff pressure is kept constant (step ST7).

Next, the stabilization detection unit 1134 detects whether the cuff pressure has stabilized as described above. If it has been detected that the cuff pressure has not stabilized, and the pressurization detection unit 1131 has detected that a pressure sensor abnormality has occurred (step ST8 and YES in step ST9), the pressure adjustment unit 111 fully opens the valve 52 via the valve driving circuit 54 (step ST10). Accordingly, air is rapidly discharged from the cuff 20, and this series of processing ends.

While the cuff pressure is stable, if the pressurization detection unit 1131 determines that a pressure sensor abnormality has not occurred (NO in step ST9), processing is performed in the same manner as the processing of steps ST14 to ST16 in FIG. 9.

In the case where abnormality detection is performed at the cuff pressures P1 and P2, an abnormality can be detected in both the pressurization process and the depressurization process.

Pressure Sensor Abnormality Detection at Cuff Pressure P3 in FIG. 5

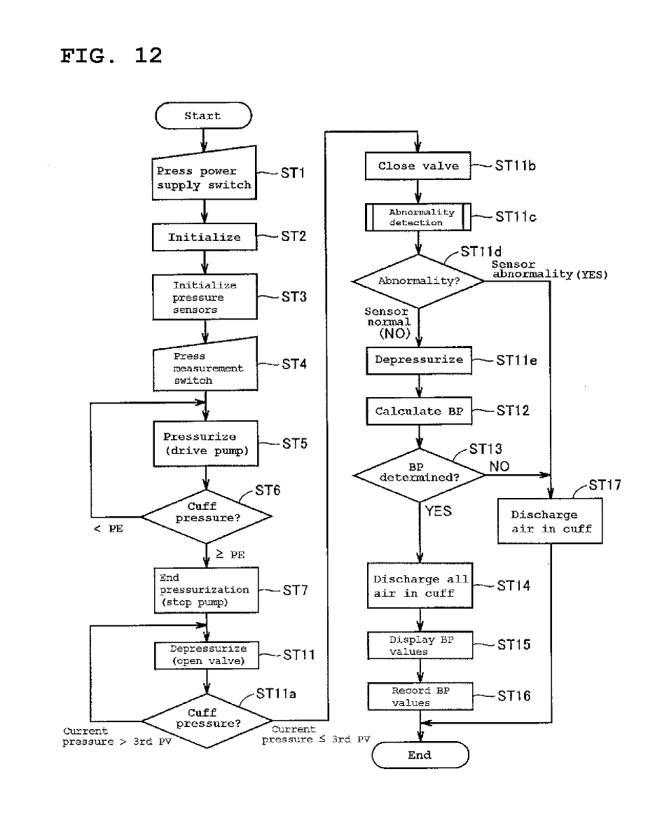

Below is a description of a procedure for carrying out pressure sensor abnormality detection at a cuff pressure (cuff pressure P3 in FIG. 5) that is higher than the systolic blood pressure SYS in the depressurization process, with reference to FIG. 12. It is assumed that the value of the cuff pressure P3 is stored in the memory 42 in advance.

In FIG. 12, the processing of steps ST1 to ST7 is performed in the same manner as the corresponding steps in FIG. 9. Next, while the pump 51 is stopped, the valve 52 is opened, and a transition is made to the depressurization process in which the cuff pressure is gradually reduced. In the depressurization process, the pressure adjustment unit 111 compares the cuff pressure detected by the first pressure sensor 321 with the cuff pressure P3 read out from the memory 42, and continues performing depressurization while it is determined, based on the comparison results, that the cuff pressure is greater than the third pressure value (i.e., the value of the cuff pressure P3), and when it has been determined that the cuff pressure is less than or equal to the third pressure value (“≦3rd PV” in step ST11a), the valve 52 closes, and the cuff pressure is kept constant (step ST11b).

Next, the stabilization detection unit 1134 detects whether the cuff pressure has stabilized as described above. If the cuff pressure has not stabilized, and the depressurization detection unit 1132 has detected that a pressure sensor abnormality has occurred (step ST11c and YES in step ST11d), the pressure adjustment unit 111 fully opens the valve 52 via the valve driving circuit 54 (step ST17). Accordingly, air is rapidly discharged from the cuff 20, and this series of processing ends. In this way, blood pressure measurement processing is stopped in the case where a pressure sensor abnormality has been detected. Note that details of abnormality detection processing (step ST11c) will be described later.

While the cuff pressure is stable, if the depressurization detection unit 1132 determines that a pressure sensor abnormality has not occurred (NO in step ST11d), the depressurization process, in which the discharge of air gradually progresses, continues, and the blood pressure is calculated during that time (steps ST11e and ST12).

If the systolic blood pressure SYS and the diastolic blood pressure DIA have been determined (YES in step ST13), processing is performed in the same manner as the processing of steps ST14 to ST16 in FIG. 9.

Pressure Sensor Abnormality Detection at Cuff Pressure P4 in FIG. 5

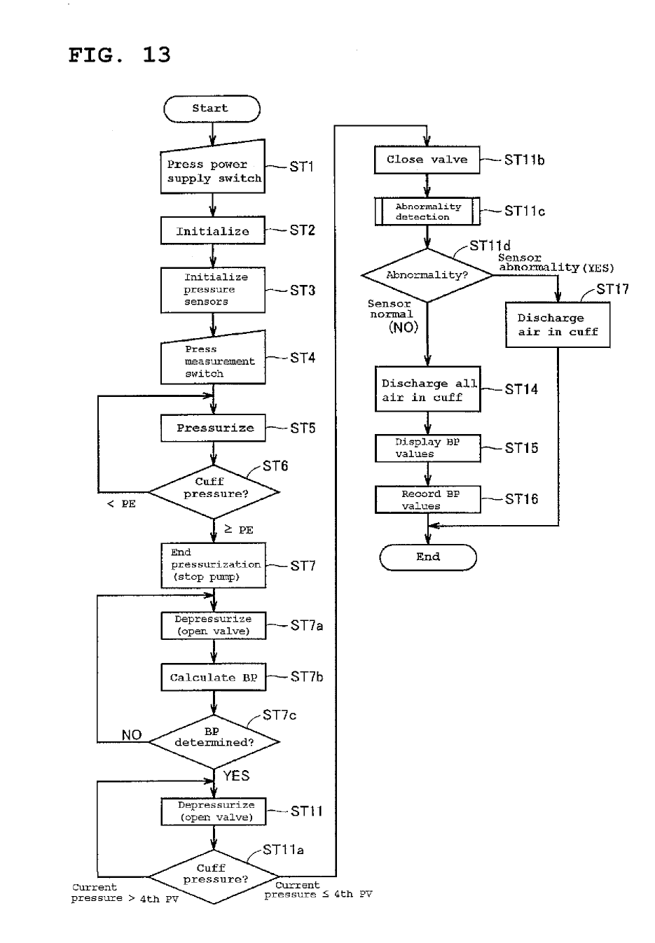

Below is a description of a procedure for carrying out pressure sensor abnormality detection at a cuff pressure (cuff pressure P4 in FIG. 5) that is lower than the diastolic blood pressure DIA in the depressurization process, with reference to FIG. 13. It is assumed that the value of the cuff pressure P4 is stored in the memory 42 in advance.

In FIG. 13, the processing of steps ST1 to ST7 is performed in the same manner as the corresponding steps in FIG. 12. Next, the valve 52 is opened while the pump 51 is stopped, thus transitioning to the depressurization process. Blood pressure calculation is performed in the depressurization process (steps ST7a to ST7c). If the blood pressure values (diastolic blood pressure DIA and systolic blood pressure SYS) have been determined, the pressure adjustment unit 111 compares the cuff pressure detected by the first pressure sensor 321 with the cuff pressure P4 read out from the memory 42, and performs depressurization until, based on the comparison results, the cuff pressure is less than or equal to the fourth pressure value (i.e., the value of the cuff pressure P4) (“≦4th PV” in step ST11a). Thereafter, the valve 52 is closed, and the cuff pressure is kept at a constant pressure (step ST11b).

Next, the stabilization detection unit 1134 detects whether the cuff pressure has stabilized as described above. If the cuff pressure has not stabilized, and the depressurization detection unit 1132 has detected that a pressure sensor abnormality has occurred (step ST11c and YES in step ST11d), the pressure adjustment unit 111 fully opens the valve 52 via the valve driving circuit 54 (step ST17). Accordingly, air is rapidly discharged from the cuff 20, and this series of processing ends.

While the cuff pressure is stable, if the depressurization detection unit 1132 determines that a pressure sensor abnormality has not occurred (NO in step ST11d), processing is performed in the same manner as the processing of steps ST14 to ST16 in FIG. 9.

In this way, pressure sensor abnormality detection is performed at least one or more timings among timings in the pressurization process, the transition period, and the depressurization process in blood pressure measurement (including the timings corresponding to the cuff pressures P1 to P4 in FIG. 5), thus eliminating the need to provide a separate abnormality detection step.

Also, in both the pressurization process and the depressurization process, abnormality detection is performed while the cuff pressure is kept constant, thus enabling obtaining relatively high detection precision.

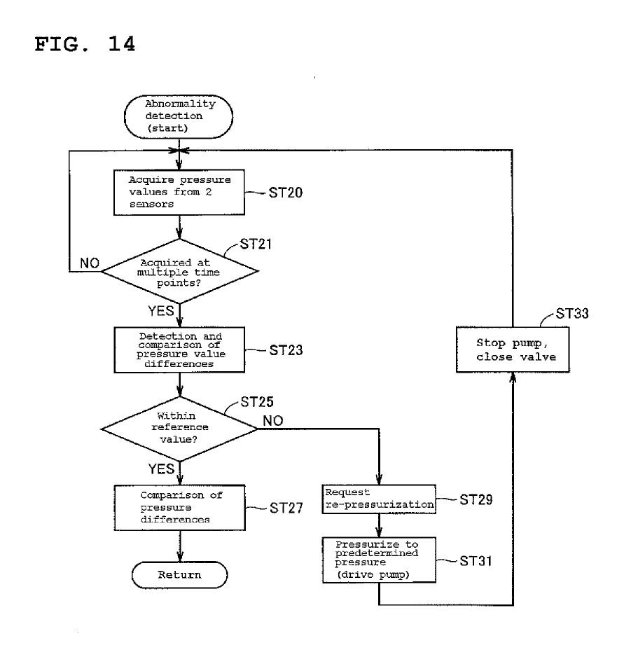

FIG. 14 is a flowchart of sensor abnormality detection processing (steps ST8 and ST11c) performed in the pressurization process and the depressurization process (before blood pressure value calculation), that is to say, at the cuff pressures P1, P2, and P3 in FIG. 5.

First, the stabilization detection unit 1134 detects cuff pressure values from the first and second pressure sensors 321 and 322 at a predetermined interval, that is to say, at multiple timings (steps ST20 and ST21). A difference between the pressures detected by the pressure sensors is then detected for each timing, and the detected differences are compared (step ST23). A determination is then made as whether the difference between the differences, which was obtained as a result of the comparison, falls within the range of the threshold value α (step ST25).

As a result of the determination, in the case where it has been detected that the difference between the differences is a value that falls within the range of the threshold value α (YES in step ST25) as shown in FIG. 7B, the abnormality detection unit 1137 detects whether each pressure difference is greater than the reference value β (step ST27). In the case where a pressure difference is detected as being greater than the reference value β, it is detected that a pressure sensor abnormality has occurred, and otherwise it is detected that a pressure sensor abnormality has not occurred.

On the other hand, in the case where it has been detected that the difference between the differences is not a value that falls within the range of the threshold value α (NO in step ST25), the re-pressurization request unit 1135 outputs a re-pressurization request signal to the pressure adjustment unit 111 (step ST29). In response to the request, the pressure adjustment unit 111 causes the pump 51 to rotate so as to raise the cuff pressure to a predetermined pressure (step ST31). When the cuff pressure reaches the predetermined pressure, the pump 51 stops, and the valve 52 closes (step ST33). After such re-pressurization, the procedure returns to the processing of step ST20, and subsequent processing is repeated.

In this way, in the case where pressure sensor abnormality detection cannot be performed because the cuff pressure stabilization period cannot be detected due to body movement, the measurement subject's pulse, or the like, the cuff pressure can be increased or reduced again so as to achieve a state in which a disturbance such as body movement or the measurement subject's pulse can be prevented, and thereafter cuff pressure stabilization detection and pressure sensor abnormality detection can be performed again.

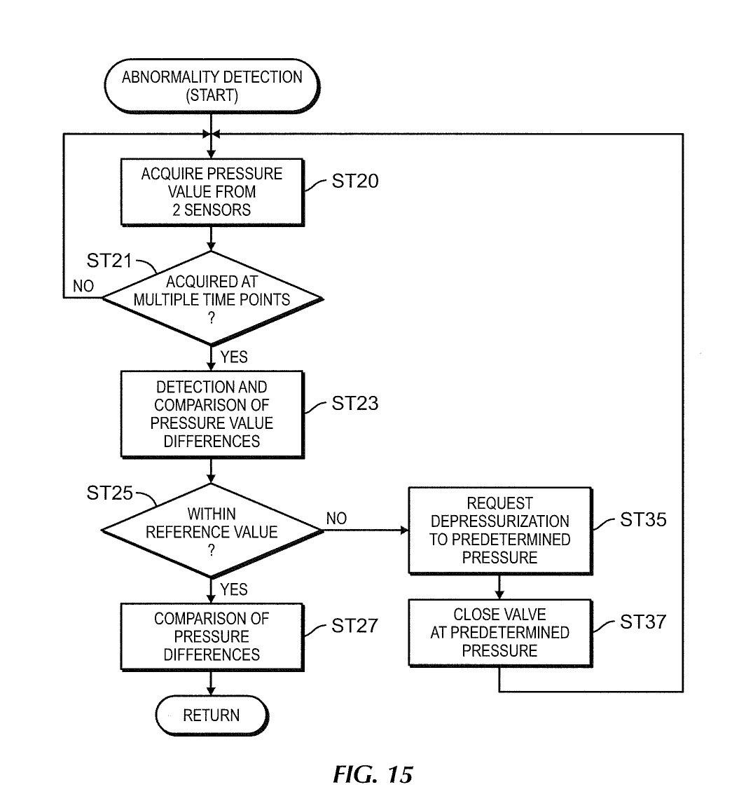

FIG. 15 is a flowchart of sensor abnormality detection processing (step ST11e) performed in the depressurization process (after blood pressure value calculation), that is to say, at the cuff pressure P4 in FIG. 5.

First, the stabilization detection unit 1134 and the abnormality detection unit 1137 perform the processing of steps ST20 to ST27 similarly to FIG. 14.

In the case where it has been detected that the difference between the differences is not a value that falls within the range of the threshold value α (NO in step ST25) as shown in FIG. 7B, the re-detection unit 1136 outputs a depressurization request signal to the pressure adjustment unit 111 (step ST35). In response to the request, the pressure adjustment unit 111 opens the valve 52 so as to reduce the cuff pressure to a predetermined pressure (step ST35). When the cuff pressure reaches the predetermined pressure, the valve 52 closes (step ST37). After such depressurization, the procedure returns to the processing of step ST20, and subsequent processing is repeated.

In this way, in the case where pressure sensor abnormality detection cannot be performed because the cuff pressure stabilization period cannot be detected due to body movement, the measurement subject's pulse, or the like, the cuff pressure can be reduced so as to achieve a state in which a disturbance such as body movement or the measurement subject's pulse can be prevented, and thereafter cuff pressure stabilization detection and pressure sensor abnormality detection can be performed again.

Display Examples

FIG. 16 shows an example of the display of pressure sensor abnormality detection results on the display unit 40. Although calculated blood pressure values are not stored in the memory 43 in the case where a pressure sensor abnormality has been detected in the flowcharts described above, the calculated blood pressure values may be stored in association with the abnormality detection result. In this case, the pressure sensor abnormality detection result is displayed along with the display of the blood pressure measurement values.

In FIG. 16, the display processing unit 115 switches the display mode based on the detection result of the sensor abnormality detection unit 113. Specifically, if the first and second pressure sensors 321 and 322 are operating normally, the display of the characters “ERR” is switched off, and only the display of the characters “OK” is switched on. If the detection result indicates that an abnormality occurred, the display of the characters “OK” is switched off, and the display of the characters “ERR” is switched on. This enables alerting the user that the apparatus is operating normally in the case where the first and second pressure sensors 321 and 322 are operating normally.

Also, an alert mode such as the following is possible. Specifically, when measurement begins, characters indicating normal operation (“OK”) are displayed, or a lamp is operated. Then, in the case where a pressure sensor abnormality has been detected, an alert regarding the abnormality may be given by causing the display of the characters or the lamp to blink. Accordingly, the alert mode when measurement starts is a mode for giving an alert regarding normal operation, and the alert mode is changed to an abnormality alert mode if an abnormality is detected.

The following are displayed on the display unit 40: measured time data 402 obtained by the measurement performed by the timer 45; systolic blood pressure SYS data 403; diastolic blood pressure DIA data 404; and pulse rate data 405, which are the results of blood pressure measurement; and “ERR”/“OK”, which indicate the result of pressure sensor abnormality detection.

By checking such a display, the measurement subject can know when to request the manufacturer to perform pressure sensor correction. This enables preventing blood pressure measurement from being performed without the measurement subject realizing that a pressure sensor abnormality has occurred, and enables improving the reliability of the blood pressure measurement values.

Note that although the electronic sphygmomanometer 1 is described in the embodiment as being a stationary electronic sphygmomanometer in which the cuff 20 is wrapped around the upper arm portion, embodiments of the present invention are not limited to this. For example, one or more embodiments of the present invention can be similarly applied to a wrist-mounted electronic sphygmomanometer in which the cuff 20 and the main body unit 10 are configured integrally, and the cuff 20 is wrapped around the wrist, as shown in FIG. 17.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### REFERENCE NUMERALS

1 electronic sphygmomanometer
321 first pressure sensor
322 second pressure sensor
331 first oscillation circuit
332 second oscillation circuit
112 blood pressure calculation unit
113 sensor abnormality detection unit

1. An electronic sphygmomanometer comprising:
a cuff that can be worn at a measurement site;
a pressure adjustment unit that adjusts a pressure inside the cuff by pressurization or depressurization;
a pressure detection unit that comprises a plurality of pressure sensors and that detects the cuff pressure inside the cuff based on pressure information output from the plurality of pressure sensors;
a blood pressure calculation unit that calculates a blood pressure based on a change in the cuff pressure detected by the pressure detection unit at a time of blood pressure measurement;
a keeping unit that keeps the cuff pressure at a predetermined pressure at the time of blood pressure measurement; and
an abnormality detection unit that, in a state in which the keeping unit keeps the cuff pressure at the predetermined pressure, detects whether an abnormality has occurred in at least one of the plurality of pressure sensors based on the pressure information output from the plurality of pressure sensors.

2. The electronic sphygmomanometer according to claim 1,
wherein the blood pressure measurement comprises:
a pressurization process in which the cuff is pressurized by the pressure adjustment unit after the blood pressure measurement has started;
a depressurization process in which the cuff is depressurized; and
a transition period from after the pressurization process is ended until when the depressurization process is started, and
wherein the keeping unit keeps the pressure applied in the cuff at the predetermined pressure in at least one of the pressurization process, the depressurization process, and the transition period.

3. The electronic sphygmomanometer according to claim 2, wherein the predetermined pressure indicates the cuff pressure at the time when the pressurization process has ended.

4. The electronic sphygmomanometer according to claim 1,
wherein the abnormality detection unit comprises:
a stabilization detection unit that detects whether the cuff pressure is being kept at the predetermined pressure based on the pressure information output from the plurality of pressure sensors, and
wherein in a case where the stabilization detection unit has detected that the cuff pressure is being kept at the predetermined pressure, the abnormality detection unit detects whether an abnormality has occurred in at least one of the plurality of pressure sensors based on the pressure information output from the plurality of pressure sensors.

5. The electronic sphygmomanometer according to claim 4, wherein the stabilization detection unit detects, with respect to the pressure information output in time series from one of the plurality of pressure sensors, a difference in the pressure information at a plurality of time points, and detects whether the cuff pressure is being kept at the predetermined pressure based on the detected difference.

6. The electronic sphygmomanometer according to claim 5, wherein the stabilization detection unit detects representative pressure information based on the pressure information output by the one of the pressure sensors at the plurality of time points, and, based on the representative pressure information, extracts, from among the pressure information at the plurality of time points output by at least one of the plurality of pressure sensors, the pressure information for detection of the difference.

7. The electronic sphygmomanometer according to claim 4, wherein based on the pressure information at a plurality of time points that has been output by the plurality of pressure sensors in time series, the stabilization detection unit detects a difference in the pressure information at each of the time points, and detects whether the cuff pressure is being kept at the predetermined pressure based on a difference between the detected differences.

8. The electronic sphygmomanometer according to claim 7, wherein the stabilization detection unit detects representative pressure information based on the pressure information output by the plurality of pressure sensors at the plurality of time points, and, based on the representative pressure information, extracts, from among the pressure information at the plurality of time points output by the plurality of pressure sensors, the pressure information for detection of the difference.

9. The electronic sphygmomanometer according to claim 1, wherein the blood pressure measurement is stopped in a case where the abnormality detection unit has detected occurrence of an abnormality in at least one of the plurality of pressure sensors.

10. The electronic sphygmomanometer according to claim 1, further comprising:
a storage unit,
wherein each time the abnormality detection unit detects whether an abnormality has occurred in at least one of the plurality of pressure sensors, the storage unit stores a result of the detection, and
wherein when the blood pressure measurement is to be started, in a case where a determination has been made that the result of the detection that was read out from the storage unit indicates that an abnormality occurred, the blood pressure measurement is stopped, and the result of the detection that was read out is output.

11. The electronic sphygmomanometer according to claim 1, wherein the electronic sphygmomanometer outputs a result of the detection performed by the abnormality detection unit.

12. The electronic sphygmomanometer according to claim 1, wherein in a case where the abnormality detection unit has detected that an abnormality occurred in at least one of the plurality of pressure sensors, the blood pressure measurement is ended, and thereafter a result of the detection performed by the abnormality detection unit is output.

13. The electronic sphygmomanometer according to claim 1, further comprising:
a data storage unit that stores blood pressure data indicating the blood pressure calculated by the blood pressure calculation unit and a result of the detection performed by the abnormality detection unit in association with the blood pressure data,
wherein among the blood pressure data in the data storage unit, the blood pressure data that is associated with a detection result indicating the occurrence of an abnormality is excluded from the blood pressure data to be used for calculation of a statistic.

14. The electronic sphygmomanometer according to claim 4, wherein in a case where the stabilization detection unit has not detected that the cuff pressure is being kept at the predetermined pressure, an alert of that fact is given.

15. The electronic sphygmomanometer according to claim 4, wherein in a case where the stabilization detection unit has not detected that the cuff pressure is being kept at the predetermined pressure, the pressure adjustment unit pressurizes the cuff, and thereafter the stabilization detection unit again detects whether the cuff pressure is being kept at the predetermined pressure.
